(19) United States
(12) Patent Application Publication
(10) Pub. No.: US 2011/0257465 A1
(43) Pub. Date: Oct. 20, 2011

(54) Stress relief harmonizing device and method

(76) Inventor: Michael Lee Landsdale, La Selva Beach, CA (US)

(21) Appl. No.: 12/799,189

(22) Filed: Apr. 20, 2010

(51) Int. Cl. A61M 21/02

(52) U.S. Cl. 600/27; 600/26

(57) ABSTRACT

The present disclosure provides a device and method for reducing stress, inducing relaxation and well-being, and increasing the ability to concentrate, by projecting a series of non-audible impulses into a selected part of a users body. The series of non-audible impulses having an ascending component and descending component where the ascending component and the descending component each including periods of breaks where there are no impulses projected onto the users body. The non-audible impulses are cued to the inhalation cycle and exhalation cycle of breath. The device may be configured as a pendent having a housing, a power source operably secured within the housing, a circuit mechanism having a plurality of different operational modes for producing the series of non-audible impulses, and at least one user operable input switch.

### BACKGROUND OF THE INVENTION

1. Technical Field

This invention relates to devices and methods for inducing relaxation and stress reduction, and more particularly, to both a portable device and method for directing a series of harmonies or vibrations into the body to induce relaxation and stress reduction, and increasing the ability to concentrate.

2. Background Art

The present invention is a device and method for inducing relaxation, stress reduction and for promoting a sense of well-being and happiness.

It has long been known and taught in many traditions of the East that the control of breath has many beneficial effects on the mind and body. These same traditions also teach that mind concentration and meditation practice are essential to well being and to promoting peace of mind and happiness.

It is also known that harmonic sounds have been used in beneficial ways to promote mind and body relaxation. Harmonic sounds and impulses projected through the skeletal system has additional beneficial effects and may be used to induce relaxation, stress reduction, and to promote a sense of well being.

In both Western and Eastern civilizations it is known and practiced that concentration on the right hemisphere of the brain develops intuitive, non-linear capacities.

There have been many books and teachings describing breathing techniques and meditation methods. The Yoga Supra written almost two-thousand years ago by Pantanjali, teaches that “We become whole by stilling the patterns of consciousness”, and later that to help do this, “Keep a close watch on the breath, outside, inside, paused or exchanged. See too the place in the body, the duration, count, and quality”. There have also been many devices and methods proposed and implemented to assist in relaxation and stress reduction. However, there has not been to applicant's knowledge, any device or methodology proposed or implemented which use a series of ascending and descending harmonic sounds or impulses cued to the human breath cycle of inhalation and exhalation, for mood enhancement, relaxation, stress reduction, and happiness.

Therefore, it would be very desirable to have a device and method that would assist a user to develop deep breathing and mindful abilities, to develop mind concentration and focus, and to enable and develop right hemisphere concentration techniques. The present device and method do all of these things while using harmonic sounds and impulese to facilitate mind and body relaxation and to project such harmonic sounds and impulses into the skeletal system of the user.

The present invention provides such a methodology and a device for implementing the method. The device as disclosed may be a portable device worn on the user's body, as a pendent or like a piece of jewelry, or on the users belt, cloths, or the like. The method may also be used as a smart phone application, for example on a smart phone such as the iPhone by Apple Computer Corp., or other electronic devices such as notebooks, notepads, and other computing and internet devices. The device and method are very useful for both instilling and teaching breathing and relaxation techniques, and to bring stress reduction and a state of well being to the user.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out in the appended claims.

### BRIEF SUMMARY OF THE INVENTION

To achieve the foregoing objects, and in accordance with the purpose of the invention as embodied and broadly described herein, The present disclosure provides a device and method for reducing stress, inducing relaxation and well-being, and increasing the ability to concentrate, by projecting a series of non-audible impulses, or harmonics, into a selected part of a users body. The series of non-audible impulses having an ascending component and descending component where the ascending component and the descending component each including periods of breaks where there are no impulses projected onto the users body. The non-audible impulses are cued to the inhalation cycle and exhalation cycle of breath. The device may be configured as a pendent having a housing, a power source operably secured within the housing, a circuit mechanism having a plurality of different operational modes for producing the series of non-audible impulses, and at least one user operable input switch. The pendent may be worn as pendent, a piece of jewelry, or attached by a clip onto pants, belt, or underwear as desired. The device is preferably battery powered and may be provided with a night stand for battery recharging.

### BRIEF DESCRIPTION OF THE DRAWINGS

The accompanying drawings, which are incorporated in and constitute a part of this specification, illustrate a preferred embodiment of the invention and, together with a general description given above and the detailed description of the preferred embodiment given below, serve to explain the principles of the invention.

FIG. 1 is a flow diagram of an exemplary embodiment of the stress relief harmonizing method, according to the invention.

FIG. 2 is a flow diagram of an exemplary embodiment of the methodology as used with a computing device, smart phone or the like, according to the invention.

FIG. 3 is an exemplary embodiment of the stress relief harmonizing device, according to the invention.

FIG. 4 is a schematic representation of the components of the device shown in FIG. 3, according to the invention.

FIG. 5 shows a smart phone, such as an Apple Computer Corp. iPhone, using the method as an application, according to the invention.

FIG. 6 shows the device having a clip for attaching the device to cloths, belt, underwear, or the like, according to the invention.

FIG. 7 shows a charging stand for charging the device, according to the invention.

### DETAILED DESCRIPTION OF THE INVENTION

Reference will now be made in detail to the present preferred embodiments of the invention as illustrated in the accompanying drawings. In the following description of the present invention, reference is made to the accompanying drawings which form a part thereof, and in which is shown by way of illustration, exemplary embodiments illustrating the principles of the present disclosure and how it may be practiced. It is to be understood that other embodiments may be utilized and structural changes may be made thereto without departing from the scope of the present disclosure.

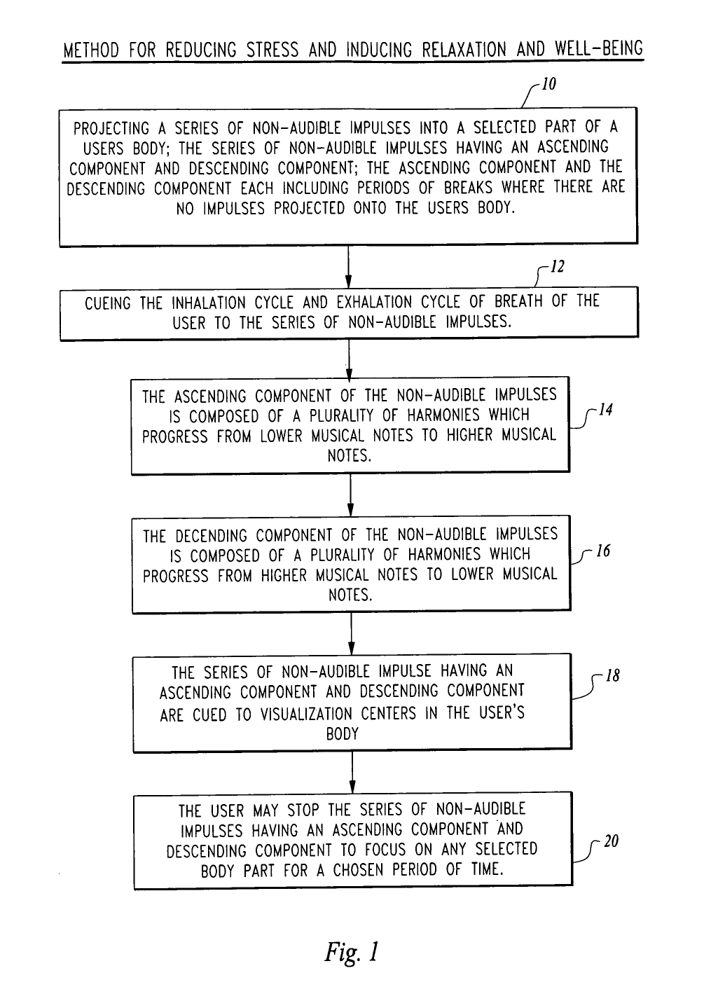

In accordance with the present invention, as seen in FIG. 1, there is provided in a preferred embodiment of the invention, a method for reducing stress and inducing relaxation and well-being. In a preferred embodiment, the method comprises first, projecting a series of non-audible impulses into a selected part of a users body; the series of non-audible impulses, or harmonics, having an ascending component and descending component; the ascending component and the descending component each including periods of breaks where there are no impulses projected onto the users body, 10. Preferably this is cued to the inhalation cycle and exhalation cycle of breath of the user to the series of non-audible impulses, 12.

Preferably, the ascending component of the non-audible impulses is composed of a plurality of harmonies which progress from lower musical notes to higher musical notes, 14. The descending component of the non-audible impulses is composed of a plurality of harmonies which progress from higher musical notes to lower musical notes, 16. The series of non-audible impulses preferably have an ascending component and descending component and are cued to visualization centers in the user's body, 18. The ascending body part visualization centers, in order are: feet, ankles, calves, knees, thighs, hips, one inch below navel, stomach, diaphragm, sternum, shoulders, upper arms, elbows, forearms, wrists, hands, where hands may be visualized projecting energy to, throat, entire head, right hemisphere of brain, forehead (third eye) and top of head (crown). These are the twenty-one ascending body part visualization centers.

The descending body part visualization centers, in order are, top of head (crown), forehead (third eye), right hemisphere of brain, entire head (visualize eyes projecting energy down to), hands, wrists, forearms, elbows, upper arms, shoulders, neck, sternum, diaphragm, stomach, one inch below navel, hips, thighs, knees, calves, ankles, and feet.

The preferred methodology also provides for that the user may stop the series of non-audible impulses having an ascending component and descending component to focus on any selected body part for a chosen period of time, 20.

With reference to FIG. 2, a block flow diagram of an exemplary embodiment of the method for inducing relaxation and well-being using a computing device is illustrated. The computing device may be a cell phone such as a smart phone, for example, the Apple Computer Corporation, iphone; computing devices such as hand-held notebook and pads, computers and the like. Preferably the method is implemented through such devices by projecting a series of non-audible multi-part harmonies into a selected part of a users body; the series of non-audible multi-part harmonies having an ascending component and descending component; the ascending component and the descending component each including periods of breaks where there are no harmonies projected onto the users body 22. Next, the series of non-audible multi-part harmonies are played on the selected part of the body, so that the multi-part harmonies are not audible to the user but may be felt in the skeletal system of the user; the series of non-audible multi-part harmonies being cued to the inhalation and exhalation cycle of the user, 24. It is preferable, to then have periods of time where the series of non-audible multi-part harmonies are stopped, this period of time being a selected period of time being cued to the inhalation and exhalation cycle of the user, 26. Preferably, the series of non-audible multi-part harmonies are projected on the user in an ascending pattern followed by the same harmonies in a descending pattern, 28.

Also seen in FIG. 2, each harmony of the series of non-audible multi-part harmonies is preferably played for six seconds, followed by a period of five seconds of silence, 30. The user is then instructed to exhale during the six seconds when the series of non-audible multi-part harmonies are played, and to exhale during said five seconds of silence, 32. The user is instructed to visualize a body part to correspond to the ascending series on non-audible multi-part harmonies, 34. The user is then instructed to visualize a body part to correspond to the descending series on non-audible multi-part harmonies, 36. As discussed above in reference to FIG. 1, the series of non-audible impulses preferably have an ascending component and descending component and are cued to visualization centers in the user's body, 18. The ascending body part visualization centers, in order are: feet, ankles, calves, knees, thighs, hips, one inch below navel, stomach, diaphragm, sternum, shoulders, upper arms, elbows, forearms, wrists, hands, where hands may be visualized projecting energy to, throat, entire head, right hemisphere of brain, forehead (third eye) and top of head (crown). These are the twenty-one ascending body part visualization centers.

The descending body part visualization centers, in order are, top of head (crown), forehead (third eye), right hemisphere of brain, entire head (visualize eyes projecting energy down to), hands, wrists, forearms, elbows, upper arms, shoulders, neck, sternum, diaphragm, stomach, one inch below navel, hips, thighs, knees, calves, ankles, and feet. The user then has the option to stop either the ascending or the descending series of non-audible multi-part harmonies on any of a plurality selected body part, 38.

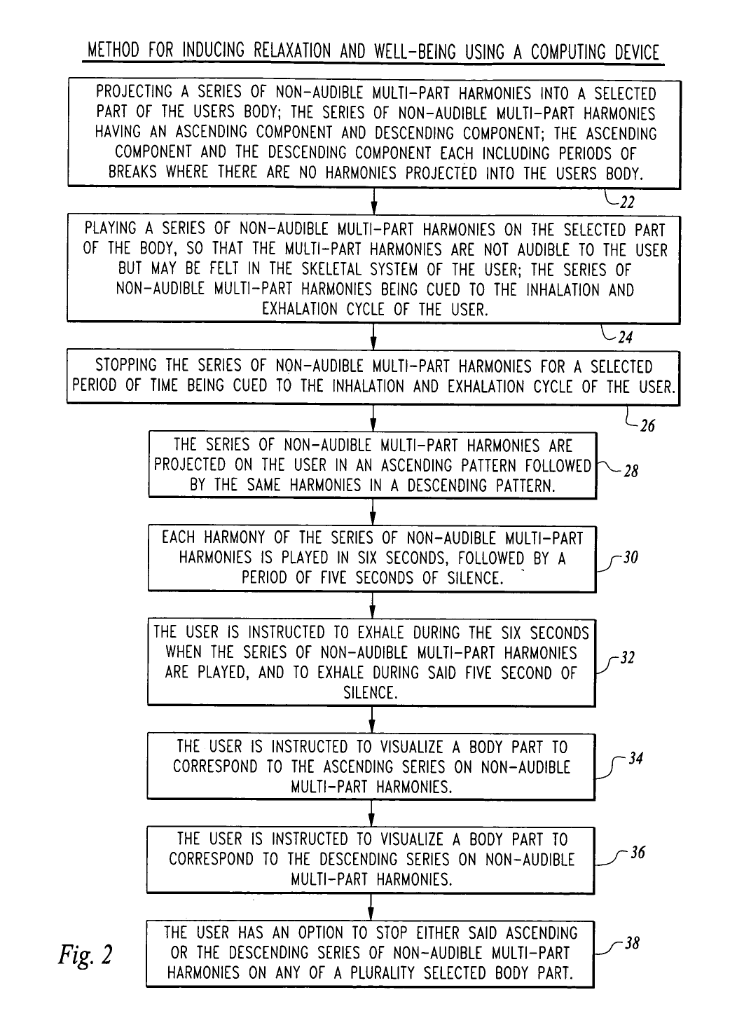

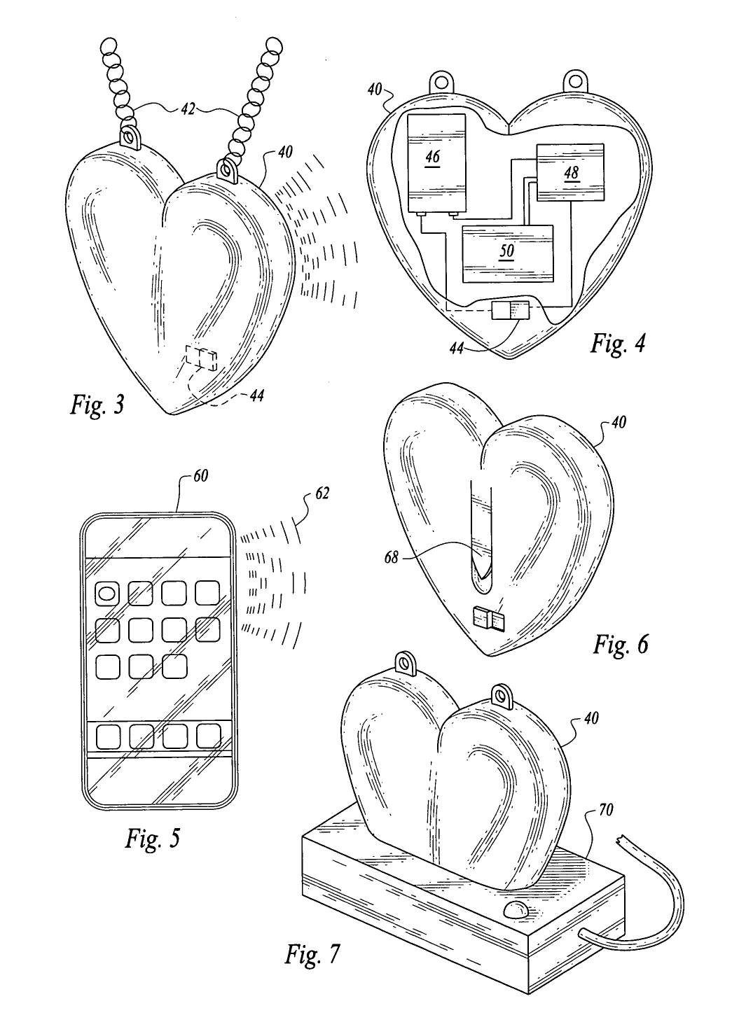

In FIG. 3, shows the portable device, in an exemplary embodiment of a pendent 40, or piece of jewelry implementing the above described methodology of the invention. Preferably the pendent has a chain, such as variable length chain 42, so the user may wear the pendent around his or her neck, and on/off switch, 44. As seen in FIG. 6, alternatively a clip, 68, may be attached to pendent 40, for providing a means for attachment of pendent 40, to cloths, belt, or underwear. This allows pendent 40, to be situated about one inch below the user's navel, an area of the body referred to in spiritual literature as the “hara” or “dan tien”.

Referring now to FIG. 4, a schematic representation of the components of the pendent 40, are shown which enable the pendent to produce the series of harmonics or non-audible impulses of the present methodology. Preferably, the pendent 40, on/off switch 44 or other activation means, operably connected to battery 40, microcontroller 48 or other control means, battery 46 as a power source, and impulses/harmonic generator 50, which may be a commercial tone generator such as the tone generator available at www.nch.com. Such tone generator is a sine wave generator, a frequency generator, and signal generator that lets the user create audio test tones, sweeps or noise waveforms. Preferably, the frequency generated is less than 20 Hertz to achieve the desired effect. However, in other embodiments a higher frequency may be used if desired. It is understood that the electrical components which govern operation of the control means may be integrated circuits capable of being designed by those skilled in the art in a wide variety of configurations. In other preferred embodiments microcontroller 48, or a microprocessor may be utilized.

Microcontroller 48, is preferably a programmable digital microcontroller, but a hardwired digital device or even an analog microcontroller may be used. The support circuitry preferably includes battery 46 as a power source. Microcontroller 48, is preferably a programmable digital controller, but may be otherwise. As is well known in the art, such programmable digital microcontrollers are small, inexpensive, and broadly available. Microcontroller 48, preferably places the arithmetic and logic controllers along with read-only memory and read-write or scratchpad memory in the chip design. Microprocessors or this type are usually utilized as embedded controllers and are programmed during manufacture.

The preferred use of microcontroller 48, in pendent 40, is that it may be programmed to provide a plurality of timing outputs while also controlling a plurality of simple output devices. Preferably the means for programming microcontroller 48, will vary according to the particular make and model being used, and are will known in the art. Microcontroller 48, in the preferred embodiment of pendent 40, is programmed according to the flow chart of FIG. 1, or FIG. 2.

In FIG. 5, a smart phone, such as an Apple Computer Corp. iPhone, is shown projecting the ascending and descending harmonies 62, as a preferred method programmed as a user application. The tone generator described above, or other tone generating software may be used in such smart phone, preferably using a frequency less that 20 Hertz to achieve the desired effect. However, in some embodiments a higher frequency may be used if desired. As described in reference to FIG. 1 and to FIG. 2, the user application methodolgy for the preferred embodiments are preferably cued to the inhalation and exhalation cycle of breath of the user. This is highly effective at both training and inducing relaxation, stress reduction, increasing the ability to concentrate, and promoting feelings of well-being.

In FIG. 7, a re-charging stand 70, for re-charging battery 46, may be provided, so the user may simply clip pendent 40, into re-charging stand 70, and re-charge the device as necessary. The particular electrical components which govern the operation of the recharging stand could be designed by those skilled in the art in a variety of configurations, and are well known in the art.

It should be understood that there are numerous other configurations that pendent 40, may be provided. It can be configured as piece of jewelry, or any other geometric design. The critical feature is that the series of non-audible impuses or harmonics, can be projected into selected areas of the human body, and cued to the inhalation and exhalation cycle of breath.

In operation and use, the present invention provides a device and method that is easy to use, reliable, and enjoyable, and that can be provided as a pendent or piece of jewelry, for example, or as an application for the user including computing devices such as smart phones, notebooks, note pads, PC, MAC, and other computing consumer devices. As described, a tone generator may be utilized or other tone generating mechanisms well known in the art to generate frequencies preferably at less than 20 Hertz, but in some embodiments a higher frequency may be used.

Additional advantages and modification will readily occur to those skilled in the art. The invention in its broader aspects is, therefore, not limited to the specific details, representative apparatus and illustrative examples shown and described. Accordingly, departures from such details may be made without departing from the spirit or scope of the applicant's general inventive concept.

What is claimed is:

1. A method for reducing stress and inducing relaxation and well-being, comprising:
projecting a series of non-audible impulses into a selected part of a users body; said series of non-audible impulses having an ascending component and descending component; said ascending component and said descending component each including periods of breaks where there are no impulses projected onto the users body; and
cueing the inhalation cycle and exhalation cycle of breath of said user to said series of non-audible impulses.

2. The method of claim 1, wherein said ascending component of said non-audible impulses is composed of a plurality of harmonies which progress from lower musical notes to higher musical notes.

3. The method of claim 1, wherein said descending component of said non-audible impulses is composed of a plurality of harmonies which progress from higher musical notes to lower musical notes.

4. The method of claim 1, wherein said series of non-audible impulses having an ascending component and descending component are cued to visualization centers in the user's body.

5. The method of claim 1, wherein the user may stop said series of non-audible impulses having an ascending component and descending component to focus on any selected body part for a chosen period of time.

6. A method for inducing relaxation and well-being using a computing device, comprising:
projecting a series of non-audible multi-part harmonies into a selected part of a users body; said series of non-audible multi-part harmonies having an ascending component and descending component; said ascending component and said descending component each including periods of breaks where there are no harmonies projected onto the users body;
playing said series of non-audible multi-part harmonies on said selected part of the body, so that said multi-part harmonies are not audible to the user but may be felt in the skeletal system of the user, said series of non-audible multi-part harmonies being cued to the inhalation and exhalation cycle of the user; and
stopping said series of non-audible multi-part harmonies for a selected period of time being cued to the inhalation and exhalation cycle of the user.

7. The method of claim 6, wherein said series of non-audible multi-part harmonies are projected on said user in an ascending pattern followed by the same harmonies in a descending pattern.

8. The method of claim 6, wherein each harmony of said series of non-audible multi-part harmonies is played for six seconds, followed by a period of five seconds of silence.

9. The method of claim 8, wherein said user is instructed to exhale during said six seconds when said series of non-audible multi-part harmonies are played, and to exhale during said five seconds of silence.

10. The method of claim 6, wherein said user is instructed to visualize a body part to correspond to said ascending series on non-audible multi-part harmonies.

11. The method of claim 6, wherein said user is instructed to visualize a body part to correspond to said descending series on non-audible multi-part harmonies.

12. The method of claim 6, wherein said user has an option to stop either said ascending or said descending series of non-audible multi-part harmonies on any of a plurality selected body part.

13. The method of claim 6, wherein said device is a smart phone.

14. A device for reducing stress and inducing relaxation and well-being, comprising:
a housing;
a power source operably secured within said housing;
a circuit means having a plurality of different operational modes for producing a series of non-audible impulses into a selected part of a users body; said series of non-audible impulses having an ascending component and descending component; said ascending component and said descending component each including periods of breaks where there are no impulses projected onto the users body; said circuit means being operably linked to said power source; and
at least one user operable input switch for enabling a user to cause said circuit means to enter a selected one of said plurality of different operational modes.

15. The device of claim 14, wherein said power source is a battery.

16. The device of claim 14, wherein said housing is secured to variable length chain.

17. The device of claim 14, wherein said housing includes a clip to secure the device onto pants, belt, clothing and the like.

18. The device of claim 14, wherein said device clips onto a stand having charging means for battery recharging.

19. The device of claim 14, wherein said device is set to provide sequentially said series of non-audible impulses having an ascending component and descending component in a selected timed series.
